SEQUENCE LISTING (23 sequences)

SEQ ID NO: 1 - DNA, 20 bp, Artificial sequence
  direct primer designed to amplify, in combination with SEQ ID NO 2, cDNA of the plexin-B1 gene
acagtgtgacaggcaaggcc

SEQ ID NO: 2 - DNA, 23 bp, Artificial sequence
  reverse primer designed to amplify, in combination with SEQ ID NO 1, cDNA of the plexin-B1 gene
cacagccaatagtgcattcaagg

SEQ ID NO: 3 - DNA, 25 bp, Artificial sequence
  probe sequence of the 33783_at of Affymetrix, the position of said probe in the mRNA sequence of the plexin-B1 gene being 6508
ttcagcctggcctgggcagccctgg

SEQ ID NO: 4 - DNA, 25 bp, Artificial sequence
  probe sequence of the 33783_at of Affymetrix, the position of said probe in the mRNA sequence of the plexin-B1 gene being 6545
gaggccaccttcttaggtgcctgta

SEQ ID NO: 5 - DNA, 25 bp, Artificial sequence
  probe sequence of the 33783_at of Affymetrix, the position of said probe in the mRNA sequence of the plexin-B1 gene being 6563
gcctgtagtgactgacaagcagagt

SEQ ID NO: 6 - DNA, 25 bp, Artificial sequence
  probe sequence of the 33783_at of Affymetrix, the position of said probe in the mRNA sequence of the plexin-B1 gene being 6565
ctgtagtgactgacaagcagagtta

SEQ ID NO: 7 - DNA, 25 bp, Artificial sequence
  probe sequence of the 33783_at of Affymetrix, the position of said probe in the mRNA sequence of the plexin-B1 gene being 6651
agacccggggcctcaaggctcatgg

SEQ ID NO: 8 - DNA, 25 bp, Artificial sequence
  probe sequence of the 33783_at of Affymetrix, the position of said probe in the mRNA sequence of the plexin-B1 gene being 6659
ggcctcaaggctcatggggtagtac

SEQ ID NO: 9 - DNA, 25 bp, Artificial sequence
  probe sequence of the 33783_at of Affymetrix, the position of said probe in the mRNA sequence of the plexin-B1 gene being 6670
tcatggggtagtacccagcctgctc

SEQ ID NO: 10 - DNA, 25 bp, Artificial sequence
  probe sequence of the 33783_at of Affymetrix, the position of said probe in the mRNA sequence of the plexin-B1 gene being 6704
agcgaccctgtgacaccggtctgca

SEQ ID NO: 11 - DNA, 25 bp, Artificial sequence
  probe sequence of the 33783_at of Affymetrix, the position of said probe in the mRNA sequence of the plexin-B1 gene being 6706
cgaccctgtgacaccggtctgcagg

SEQ ID NO: 12 - DNA, 25 bp, Artificial sequence
  probe sequence of the 33783_at of Affymetrix, the position of said probe in the mRNA sequence of the plexin-B1 gene being 6809
ctggccttggccacactgggattcg

SEQ ID NO: 13 - DNA, 25 bp, Artificial sequence
  probe sequence of the 33783_at of Affymetrix, the position of said probe in the mRNA sequence of the plexin-B1 gene being 6812
gccttggccacactgggattcggag

SEQ ID NO: 14 - DNA, 25 bp, Artificial sequence
  probe sequence of the 33783_at of Affymetrix, the position of said probe in the mRNA sequence of the plexin-B1 gene being 6843
gaggagagccccatgcttcctgtct

SEQ ID NO: 15 - DNA, 25 bp, Artificial sequence
  probe sequence of the 33783_at of Affymetrix, the position of said probe in the mRNA sequence of the plexin-B1 gene being 6845
ggagagccccatgcttcctgtctgc

SEQ ID NO: 16 - DNA, 25 bp, Artificial sequence
  probe sequence of the 33783_at of Affymetrix, the position of said probe in the mRNA sequence of the plexin-B1 gene being 6997
acagggctgccctgcctcataggta

SEQ ID NO: 17 - DNA, 25 bp, Artificial sequence
  probe sequence of the 33783_at of Affymetrix, the position of said probe in the mRNA sequence of the plexin-B1 gene being 7009
tgcctcataggtagccatggtgagg

SEQ ID NO: 18 - DNA, 25 bp, Artificial sequence
  probe sequence of the 33783_at of Affymetrix, the position of said probe in the mRNA sequence of the plexin-B1 gene being 7061
agagtggtgactccattgacccagc

SEQ ID NO: 19 - DNA, 21 bp, Artificial sequence
  direct primer designed to amplify, in combination with SEQ ID NO 20, a fragment of human plexin-B1 located at the 3′end of the coding sequence
tcaacgcggacagttcaagta

SEQ ID NO: 20 - DNA, 20 bp, Artificial sequence
  reverse primer designed to amplify, in combination with SEQ ID NO 19, a fragment of human plexin-B1 located at the 3′end of the coding sequence
cacggacgcatatctcacgt

SEQ ID NO: 21 - DNA, 17 bp, Artificial sequence
  direct primer designed to amplify, in combination with SEQ ID NO 22, a fragment of rib I10 gene used as a control in the RT-PCR reaction
tgcgatggctgcacaca

SEQ ID NO: 22 - DNA, 23 bp, Artificial sequence
  reverse primer designed to amplify, in combination with SEQ ID NO 21, a fragment of rib I10 gene used as a control in the RT-PCR reaction
tcccttagagcaacccatacaac

SEQ ID NO: 23 - protein, 15 aa, Artificial sequence
  Peptide containing residues 1113-1127 of human plexin-B1
CAVDAQEYEVSSSLV

(19) United States
(12) Patent Application Publication
(10) Pub. No.: US 2008/0015143 A1
(43) Pub. Date: Jan. 17, 2008

(54) In Vitro Methods for Detecting Renal Cancer

(76) Inventors: Jose Javier Gomez Roman, Santander (ES); Maria Pilar Saenz Jimenez, Derio (ES); Jorge Ochoa Garay, Derio (ES); Jokin Del Amo Iribarren, Derio (ES); Cristina Sanz Ibayondo, Santander (ES); Corina Junquera Sanchez-Vallejo, Derio (ES); Laureano Simon Buela, Derio (ES); Antonio Martinez Martinez, Derio (ES); Jose Fernando Val Bernal, Santander (ES); Jorge Cuevas-Gonzalez, Leon (ES)

Correspondence Address: INTELLECTUAL PROPERTY / TECHNOLOGY LAW, PO BOX 14329, RESEARCH TRIANGLE PARK, NC, 27709, US

(21) Appl. No.: 10/563,025

(22) PCT Filed: Jun. 30, 2004
(86) PCT No.: PCT/EP04/07195

§ 371 Date: Aug. 3, 2007

(30) Foreign Application Priority Data
Jun. 30, 2003 (ES) 200301518

(51) Int. Cl. G01N 33/574; A61K 31/7088; A61K 38/16; C12N 15/00; G01N 33/68; C12Q 1/68; G01N 33/566

(52) U.S. Cl. 514/12; 435/29; 435/320.1; 435/6; 435/7.23; 435/7.92; 435/7.93; 435/7.94; 436/501; 436/86; 436/94; 514/44

(57) ABSTRACT

The present invention refers to an in vitro method for detecting the presence of renal cancer in an individual, for determining the stage, malignancy or severity of said carcinoma in the individual or for monitoring the effect of the therapy administered to an individual having said cancer; to the search, identification, development and assessment of the efficacy of compounds for therapy for said cancer in an attempt to develop new drugs; as well as to agents inducing Plexin-131 protein expression and/or activity, or to agents inhibiting the effects of Plexin-B1 protein expression and/or activity repression.

### FIELD OF THE INVENTION

The present invention refers to an in vitro method for detecting the presence of renal cancer in an individual, for determining the stage, malignancy or severity of said carcinoma in the individual or for monitoring the effect of the therapy administered to an individual having said cancer; to the search, identification, development and assessment of the efficacy of compounds for therapy for said cancer in an attempt to develop new drugs; as well as to agents inducing Plexin-B1 protein expression and/or activity, or to agents inhibiting the effects of Plexin-B1 protein expression and/or activity repression.

### BACKGROUND OF THE INVENTION

Despite all the advances which have been made in recent years, cancer is still one of the main causes of death worldwide. More than 5 million new cancer cases are diagnosed worldwide each year, and more than 3.5 million deaths are caused by several types of cancer, according to data provided by the International Agency for Research on Cancer, GLOBOCAN, from the year 2000.

Renal neoplasms are frequent tumors in western societies; they represent 2% of all cancer cases. According to data from the International Agency for Research on Cancer, GLOBOCAN, from the year 2000, more than 90,000 new cases are diagnosed each year in Europe, 9,000 in Japan and 30,000 in North America; more than 39,000, 4,000 and 12,000 deaths are caused each year by renal carcinoma in Europe, Japan and North America, respectively; and worldwide mortality caused by renal carcinoma exceeds 100,000 cases per year.

Advances in knowledge of cancer genetics have allowed for the classification of several types of renal tumors. The most common subtype is the conventional renal cell carcinoma, which is different from the papillary, chromophobe or collecting duct subtypes. Renal oncocytoma is a benign neoplasm at times undistinguishable from renal carcinoma, which is a malignant neoplasm. Prior studies have shown that all these histological subtypes are genetically and biologically different, and that both their morphology and behavior are determined by distinctive molecular factors (Kovacs G., et al., J. Pathol., 1997, 183:131-133).

Renal cancer diagnosis systems are currently based on the identification of clinical symptoms and on radiological techniques. The most common symptoms are hematuria (in 50-60% of patients), abdominal pain (in 40% of patients) and a palpable mass in the flank of the abdomen (in 30-40% of patients) (Ritchie A. W. S. and Chisholm G. D., Semin. Oncol., 1983, 10:390-400). The simultaneous combination of these symptoms (“classic triad”) occurs in less than 10% of patients. Small, localized tumors rarely cause symptoms, delaying diagnosis until advanced stages. 25% of the tumors are diagnosed by means of computer tomography and ultrasonography (Porena M., et al., J. Clin. Ultrasound, 1992, 20:395-400; Konnack J. W. and Grossman H. B., J. Urol., 1985, 134:1094-1096; Thompson I. M. and Peek M., J. Urol., 1988, 140:487-490); tumors diagnosed with these techniques are usually smaller than those causing symptoms and are more easily resectable to achieve healing (Konnack J. W. and Grossman H. B., J. Urol., 1985, 134:1094-1096; Thompson I. M. and Peek M., J. Urol., 1988, 140:487-490; Smith S. J., et al., Radiology, 1989, 170:699-703).

Alteration of gene expression levels is closely related to uncontrolled cell growth and to de-differentiation processes, common occurrences in all types of cancer. Expression levels of the so-called “tumor suppressing genes”, which act to prevent malignant cell growth, are repressed in the tumor cells; and expression levels of the so-called “oncogenes”, which act to induce malignant growth, are higher in tumor cells.

Many genes have been associated to the development of renal cancer, such as the von Hippel-Lindau gene (Seizinger B. R., et al., Nature, 1988, 332:268-269), the epidermal growth factor receptor gene (Ishikawa J., et al., Int. J. Cancer, 1990, 45:1018-1021), the transforming growth factor-alpha gene (Lager D. J., et al., Mod. Pathol., 1994, 7:544), the c-myc oncogene (Yao M., et al., Cancer Res., 1988, 48:6753-6757), retinoid metabolism genes (Guo X., et al., Cancer Res., 2001, 61:2774-2781), or cell adhesion factor genes (Kuroiwa, K., et al., J. Surg. Oncol., 2001, 77:123-131). However, many of the genes involved in the beginning and in the progression of renal tumors are still unknown.

There is currently no non-invasive in vitro method detecting renal tumors with acceptable sensitivity and specificity levels; a highly sensitive and specific non-invasive method would allow for routinely carrying out clinical analyses for detecting these tumors. The identification of genes differentially expressed in renal carcinomas could allow for the identification of biological markers, which could be highly valuable for the in vitro diagnosis, in vitro prognosis and treatment of this disease (Boer J. M., et al., Genome Res., 2001, 11:1861-1870). The detection of changes in the gene expression level or in the concentration of encoded proteins in body fluids could be the basis of non-invasive in vitro diagnosis renal cancer methods.

Once the patient has been diagnosed with renal cancer, surgical resection is the treatment of choice. Radical nephrectomy, including resection of the kidney, perirenal fat and the ipsilateral adrenal gland, has generally been carried out since the 1960s (Robson C. J., et al., J. Urol, 1969, 101:297-301). Nephron-sparing surgery has been used to treat small, localized tumors (Herr H. W., Cancer, 1994, 73:160-162). The most important parameter for predicting survival is the anatomical extension of the tumor. Patients with small, localized tumors which are completely resected have higher survival rates than those having affected nodes or distal metastasis. 20-30% of patients with localized tumors recover after radical nephrectomy; less than 5% experience local recidivations and 50-60% of patients develop distant metastasis (Rabinovitch R. A., et al., J. Clin. Oncol., 1994, 12:206-212; Sandock, D. S., et al., J. Urol, 1995, 154:28-31). Once metastasis develops, the long-term survival prognosis significantly worsens; currently, 30% of patients diagnosed are already in stage IV, i.e. they have developed distant metastasis.

Treating renal carcinoma is extremely difficult due to its capability of extending without causing symptoms, due to its inherent resistance to conventional systemic chemotherapy and due to the inability of radiotherapy to reduce post-nephrectomy relapse levels, even in patients with affected nodes or tumors not completely resected (Kjaer M., et al., Int. J. Radiat. Oncol. Biol. Phys., 1987, 13:665-672). Almost 50% of patients, and 90-95% of patients with distant metastasis, die during the five years after diagnosis. To combat this reality, molecular genetics studies are providing knowledge of the pathogenesis of this disease and they can provide new targets against which new therapeutic agents can be developed which are more effective than those currently available.

The family of proteins called Plexins is made up of several proteins with cellular cytoplasmic transmembrane domains; Plexins act as semaphorin protein family receptors, although they have also been found expressed in extraneuronal tissues with other molecular functions. Plexin-B1 acts as a receptor of semaphorin III, which is a protein causing neuronal growth cone collapse and the chemical repulsion of axons (Driessens M. H., Olivo C., Nagata K., Inagaki, M., Collard J. G., FEBS lett., 2002, 529:168-172). As of today it has not been proven that Plexins play a role in the carcinogenesis process; however it has been proven that there is a complex and dynamic pattern of interaction between Plexins and small guanosine triphosphatase rho proteins (Castellani V., et al., Curr. Opin. Neurobiol., 2002, 12:532). These Rho-GTPases have a function in reordering the cytoskeleton and are involved in cell adhesion and migration processes; in fact, activation of the Rac protein, one of the members of this family, has been associated with cell migration induction processes and the development of a tumor invasion phenotype (Keely, et al., 1997. Nature 390, 632-637; Sander, et al., J. Cell Biol., 1998, 143:1385-1398). On the other hand, p21 protein-activated kinase (PAK) is the Rac activating factor and competes with Plexin-B1 in order to bind to Rac; this interaction is two-way given that the Plexin-B1—Rac interaction inhibits PAK activation (Vikis H. G., Li W., and Guan K. L. Genes Dev., 2002, 16:836-845; Vikis H. G., Li W., and Guan K. L. Proc. Natl. Acad. Sci. USA, 2002, 99:12085-12090). It has also been disclosed, using DNA chips, that the plexin-B1 gene was one of the 213 genes having reduced expression in renal cell carcinoma samples when compared with healthy kidney samples (Gieseg M. A., Cody T., Man M. Z., Madore S. J., Rubin M. A., and Kalfian E. P., BMC Bioinformatics, 2002, 3:26); however, the authors of this work did not prove that the differential gene expression levels of Plexin-B1 were maintained when analyzing the samples by methods which are more sensitive, specific and reliable than DNA-chips, such as by quantitative RT-PCR for example; nor did they prove that the differential gene expression levels translated into differential protein expression levels.

Consistent with this hypothetical function of Plexin-B1, after laborious research and by using different techniques (DNA-chips and quantitative PCR to measure gene expression levels and Western-blot to measure protein expression levels), the authors of the present invention have discovered that interestingly plexin-B1 gene expression is reduced in renal carcinomas, the reduction being proportional to malignity and invasiveness. Also, the authors of the present invention have discovered that the concentration of the Plexin-B1 protein is also reduced in renal carcinomas. Finally, and yet more interestingly, the authors of the present invention have discovered that the overexpression of Plexin-B1 causes a decrease in cell proliferation of renal carcinoma cells.

The present invention therefore provides an in vitro method for detecting the presence of a carcinoma in an individual, for determining the stage or severity of said carcinoma in the individual or for monitoring the effect of the therapy administered to an individual having said carcinoma, based on the detection and/or quantification of the Plexin-B1 protein, of the plexin-B1 gene mRNA or the corresponding cDNA in a sample from said individual. The present invention also provides a method for treating renal cancer comprising the administration of Plexin-B1 protein.

### SUMMARY OF THE INVENTION

A first aspect of the present invention is to provide an in vitro method to detect the presence of renal cancer in an individual, to determine the stage or severity of said cancer in the individual or to monitor the effect of the therapy administered to an individual with this cancer.

A second aspect of the present invention is an in vitro method to screen for, identify, develop and evaluate the efficacy of compounds to treat renal cancer.

An additional aspect of the invention lies in the use of sequences derived from the plexin-B1 gene in methods to establish the diagnosis and prognosis in vitro of renal cancer, and to screen for, identify, develop and evaluate the efficacy of compounds for the treatment of renal cancer.

A further aspect of the present invention consists in the provision of agents that induce Plexin-B1 protein expression and/or activity, or in that they inhibit the carcinogenic effects of the repression of plexin-B1 protein expression, for treating renal cancer.

Another aspect of the present invention consists in a pharmaceutical composition comprising one or several therapeutic agents together with a pharmaceutically acceptable excipient for treating kidney cancer. Preferably, one of these agents is the Plexin-B1 protein.

A final aspect of the present invention consists in a kit for carrying out the present invention.

### DESCRIPTION OF THE DRAWINGS

FIG. 1 shows differential plexin-B1 gene expression levels, by real time quantitative RT-PCR, in samples from kidney biopsies of individuals affected with different types of renal carcinomas. The bars represent the plexin-B1 gene expression level compared to the gadph gene (reference gene) expression level in each one of the tumor and non-tumor samples.

FIG. 2 shows that the growth rate of plexin-B1 expressing clones (AP2-AP12) is slower than the growth of the control clone (AM2) or that of parenteral ACHN cells. The growth rate is represented by B in y=A·e^{Bx}, the equation that describes the regression curve of exponentially growing cultures.

### DETAILED DESCRIPTION OF THE INVENTION

To facilitate understanding of the present patent application, the meaning of some terms and expressions in the context of the invention will be explained below:

The terms “subject” or “individual” refer to members of mammalian animal species and includes, but is not limited to, domestic animals, primates and humans; the subject is preferably a human being, male or female, of any age or race.

The term “cancer or carcinoma” refers to the disease which is characterized by an uncontrolled proliferation of abnormal cells capable of invading adjacent tissues and spreading to distant organs.

The term “renal cancer” refers to any proliferative malignant disorder of kidney cells.

The term “tumor” refers to any abnormal tissue mass resulting from a benign (non-cancerous) or malignant (cancerous) neoplastic process.

The term “gene” refers to a molecular chain of deoxyribonucleotides encoding a protein.

The term “DNA” refers to deoxyribonucleic acid. A DNA sequence is a deoxyribonucleotide sequence.

The term “cDNA” refers to a nucleotide sequence complementary of an mRNA sequence.

The term “RNA” refers to ribonucleic acid. An RNA sequence is a ribonucleotide sequence.

The term “mRNA” refers to messenger ribonucleic acid, which is the fraction of total RNA which is translated into proteins.

The phrase “mRNA transcribed from” refers to the transcription of the gene (DNA) into mRNA as a first step for the gene to be expressed and translated to a protein.

The term “sequence of nucleotides” or “nucleotide sequence” indistinctly refers to a ribonucleotide (RNA) or deoxyribonucleotide (DNA) sequence.

The term “protein” refers to a molecular chain of amino acids with biological activity.

The “plexin-B1” gene corresponds to the gene called “plexin-B1” in English and has GeneBank code number ACCB007867.

The terms “peptide” and “polypeptide” refer to a protein fragment. The terms “protein” and “peptide” are indistinctly used.

The phrase “reduced expression” means that the measured gene or protein levels in cancer patients are lower than the levels measured in a control population of subjects with no history of cancer.

The term “sensitivity” refers to the detection of false negatives (negative diagnosis of cancer when the patient has cancer); 100% sensitivity means that there are no false negatives.

The term “specificity” refers to the detection of false positives (positive diagnosis of cancer when the patient does not have cancer); 100% specificity means that there are no false positives.

The term “antibody” refers to a glycoprotein exhibiting specific binding activity to a particular protein, which is called “antigen”. The term “antibody” comprises monoclonal antibodies, or polyclonal antibodies, either intact or fragments thereof, and includes human, humanized and non-human origin antibodies. “Monoclonal antibodies” are homogenous populations of highly specific antibodies directed against a single site or antigenic “determinant”. “Polyclonal antibodies” include heterogeneous populations of antibodies directed against different antigenic determinants.

The term “epitope”, as it is used in the present invention, refers to an antigenic determinant of a protein, which is the amino acid sequence of the protein recognized by a specific antibody.

The term “solid phase”, as it is used in the present invention, refers to a non-aqueous matrix to which the antibody can bind. Examples of solid phase materials include glass, polysaccharides, for example agarose, polyacrylamide, polystyrene, polyvinyl alcohol and silicones. Examples of solid phase forms are the well of an assay plate or a purification column.

The term “oligonucleotide primer”, as it is used in the present invention, refers to a nucleotide sequence that is complementary of a nucleotide sequence of the plexin-B1 gene. Each primer hybridizes with its target nucleotide sequence and acts as a DNA polymerization initiation site.

The term “probe”, as it is used in the present invention, refers to a nucleotide sequence that is complementary of a nucleotide sequence derived from the plexin-B1 gene, which can be used to detect that nucleotide sequence derived from the plexin-B1 gene.

The term “therapeutic target” refers to nucleotide or peptide sequences against which a therapeutic compound or drug can be designed and clinically applied.

The term “agonist” refers to any molecule mimicking the biological activity of the agonized molecule. Examples of agonist molecules include proteins, peptides, natural peptide sequence variations and small organic molecules (molecular weight lower than 500 daltons), among others.

The term “recombinant expression vector” refers to a replicon to which another nucleotide sequence is bound so that this other sequence can be transported, introduced and expressed inside the cells.

The term “replicon” refers to a nucleotide sequence able to self-replicate inside cells.

The present invention is based on the discovery that both the gene expression of the plexin-B1 gene and the concentration of the plexin-B1 protein are repressed with the development of renal cancers.

In this sense, the present invention first of all provides an in vitro method that comprises:
a) the detection and/or quantification of the Plexin-B1 protein, of the mRNA of the plexin-B1 gene, or of the corresponding cDNA in a sample of an individual, and
b) the comparison of the amount of Plexin-B1 protein, of the amount of plexin-B1 gene mRNA or of the amount of the corresponding cDNA detected in a sample from an individual, with their normal reference values.

Said in vitro method is employed to detect the presence of renal cancer in an individual, to determine the stage or severity of this cancer in an individual or to monitor the effect of the therapy administered to the individual with this cancer.

The method provided by the present invention is highly sensitive and specific and is based on subjects or individuals diagnosed with kidney cancer, having low levels of plexin-B1 gene transcribed mRNA, reduced plexin-B1 gene expression levels or reduced concentrations of the protein encoded by the plexin-B1 gene (the Plexin-B1 protein), in comparison with the corresponding levels in samples from subjects with no clinical history of cancer, such as renal cancer for example.

The present method comprises a step of obtaining the sample from the individual. Different fluid samples, such as urine, blood, plasma, serum, pleural fluid, ascitic fluid, synovial fluid, bile, semen or cerebrospinal fluid, for example, can be worked with. The sample can also be a tissue, such as kidney tissue, for example, which can be obtained by any conventional method, preferably nephrectomy. Samples can be obtained from subjects previously diagnosed or not diagnosed with renal cancer, or also from a subject undergoing treatment, or who has previously been treated for renal cancer.

The present method also comprises a step for extracting the sample, either for obtaining the protein extract of the sample or for obtaining the total RNA extract. One of these two extracts represents the working material for the next phase. Total protein or RNA extraction protocols are well known by a person skilled in the art (Chomczynski P. et al., Anal. Biochem., 1987, 162: 156; Chomczynski P., Biotechniques, 1993, 15: 532). Any conventional assay can be used within the framework of the invention for detecting renal cancer, as long as they perform the in vitro measurement of plexin-B1 gene transcribed mRNA or its complementary cDNA or the Plexin-B1 protein concentration in samples taken from the individuals to be analyzed and from control individuals.

Therefore, this invention provides a method to detect the presence of renal cancer in an individual, to determine the stage or severity of this cancer in an individual, or to monitor the effect of the therapy administered to an individual who presents this cancer, based either on measuring the levels of the Plexin-B1 protein or on measuring the level of expression of the Plexin-B1 gene.

In the event that the protein is to be detected, specifically the Plexin-B1 protein, the method of the invention comprises a first step in which the protein extract of the sample is placed in contact with a composition of one or more specific antibodies against one or more epitopes of the Plexin-B1 protein, and a second step, in which the complexes formed by the antibodies of the Plexin-B1 protein are quantified.

There is a wide variety of immunological assays available for detecting and quantifying the formation of specific antigen-antibody complexes; numerous competitive and non-competitive protein binding assays have been previously disclosed, and a large number of these assays are available on the market. Therefore, the Plexin-B1 protein can be quantified with antibodies such as, for example: monoclonal antibodies, polyclonal antibodies, either intact or recombinant fragments thereof, combined antibodies and Fab or scFv antibody fragments, specific against the Plexin-B1 protein; these antibodies being human, humanized or of a non-human origin. There are antibodies specifically binding to the Plexin-B1 protein which are available on the market. The antibodies used in these assays may be marked or not; the unmarked antibodies can be used in agglutination assays; the marked antibodies can be used in a wide variety of assays. The marker molecules which can be used for marking the antibodies include radionucleotides, enzymes, fluorophores, chemiluminescent reagents, enzyme substrates or cofactors, enzyme inhibitors, particles, dyes and derivatives. There is a wide variety of well known assays which can be used in the present invention using unmarked antibodies (primary antibody) and marked antibodies (secondary antibody); included among these techniques are Western-blot, ELISA (Enzyme-Linked immunosorbent assay), RIA (Radioimmunoassay), competitive EIA (Competitive enzyme immunoassay), DAS-ELISA (Double antibody sandwich-ELISA), immunocytochemical and immunohistochemical techniques, techniques based on the use of protein biochips or microarrays including specific antibodies or assays based on colloidal precipitation in formats such as dipsticks. Other ways of detecting and quantifying the Plexin-B1 protein include affinity chromatography techniques, ligand binding assays or lectin binding assays. The preferred immunoassay in the method of the invention is a double antibody sandwich ELISA assay. Any antibody or combination of antibodies specific against one or more epitopes of the Plexin-B1 protein can be used in this immunoassay. As an example of one of the many possible formats of this assay, a monoclonal or a polyclonal antibody, or a fragment of this antibody, or a combination of antibodies, coating a solid phase are placed in contact with the sample to be analyzed and are incubated for a time and under conditions which are suitable for forming the antigen-antibody complexes. An indicator reagent comprising a monoclonal or polyclonal antibody, or a fragment of this antibody, or a combination of these antibodies, bound to a signal generating compound is incubated with the antigen-antibody complexes for a suitable time and under suitable conditions after washing under suitable conditions for eliminating the non-specific complexes. The presence of the Plexin-B1 protein in the sample to be analyzed is detected and quantified, in the event that it exists, by measuring the generated signal. The amount of Plexin-B1 protein present in the sample to be analyzed is proportional to that signal.

In the event that the mRNA or cDNA corresponding to the plexin-B1 gene and not the protein is to be detected, the method of the invention has also several different steps. Therefore, once the sample has been obtained and the total RNA has been extracted, the method of the invention, the detection of mRNA or of the corresponding cDNA of the plexin-B1 gene, comprises a first step of amplification of the total RNA extract or of the corresponding cDNA synthesized by reverse transcription of the mRNA, and a second step of quantification of the amplification product of the plexin-B1 gene mRNA or cDNA. One example of mRNA amplification consists of reverse transcribing (RT) the mRNA into cDNA, followed by the Polymerase Chain Reaction (PCR) using oligonucleotide primers, the sequences of the primers used being SEQ ID NO.1 and SEQ ID NO.2. PCR is an amplification technique for amplifying a determined nucleotide sequence (target) contained in a mixture of nucleotide sequences. An excess pair of oligonucleotide primers is used in PCR, which hybridize with the complementary strands of the target nucleotide sequence. Then, an enzyme with polymerase activity (Taq DNA Polymerase) extends each primer, using the target nucleotide sequence as a mold. The products of the extension are then converted into target sequences after the disassociation of the original target strand. New primer molecules hybridize, and the polymerase extends them; the cycle is repeated in order to exponentially increase the number of target sequences. This technique is disclosed in U.S. Pat. Nos. 4,683,195 and 4,683,202. Many methods for detecting and quantifying the PCR amplification products have been previously disclosed, any of which methods could be used in this invention. In a preferred method of the invention, the amplified product is detected by electrophoresis in agarose gel in the following manner: five microliters of the amplification product are subjected to an electrophoresis separation in agarose gel at a 2% concentration in a 0.5×TBE buffer at 100 vdc for one hour. After electrophoresis, the gel is stained with ethidium bromide and the amplification product can be visualized by illuminating the gel with ultraviolet (UV) light; as an alternative to staining, and a preferred embodiment, the amplified product can be blotted onto a nylon membrane by means of Southern blotting techniques, to be detected with a specific probe of the cDNA of the suitably marked plexin-B1 gene. In another example, mRNA detection is carried out by blotting the mRNA onto a nylon membrane by means of blotting techniques, such as the Northern blot, for example, and detecting it with specific probes of the mRNA or of the corresponding cDNA of the plexin-B1 gene. In a particular embodiment, the amplification and quantification of the mRNA corresponding to the plexin-B1 gene is carried out by means of real time quantitative RT-PCR (Q-PCR).

The final step of the method of the invention consists of comparing the amount of Plexin-B1 protein, the amount of plexin-B1 gene mRNA or the amount of the corresponding cDNA detected in a sample from an individual, with the amount of Plexin-B1 protein, the amount of plexin-B1 gene mRNA or the amount of the corresponding cDNA detected in samples from control subjects or in previous samples from the same individual, or with normal reference values.

The method also provides an in vitro method for identifying and assessing the efficacy of therapeutic agents for renal cancer therapy comprising:
a) placing an immortalized kidney cell culture, in contact with the candidate agent under the conditions and for the time suitable for allowing them to interact,
b) detecting and quantifying the plexin-B1 gene or the Plexin-B1 protein expression levels, and
c) comparing said expression levels with those of immortalized kidney cell control cultures not treated with the candidate compound.

The quantification of the plexin-B1 gene or the Plexin-B1 protein expression levels is carried out in a manner similar to that indicated in the method of the invention for detecting in vitro the presence of kidney cancer in an individual.

When an agent increases the plexin-B1 gene expression levels or reverts the effects of the reduced expression of said gene, preferably decreasing cell proliferation levels, this agent becomes a candidate for cancer therapy, and especially for renal carcinoma.

Another aspect of the invention refers to the use of nucleotide or peptide sequences derived from the plexin-B1 gene for the search, identification, development and assesment of the efficacy of compounds for renal cancer therapy. It is noteworthy, the recent importance given to screening methods based on the competitive or non-competitive binding of the potential therapeutic molecule to therapeutic target.

In another aspect of the invention reference is made to agents that induce Plexin-B1 protein expression and/or activity, or that in inhibit the effects of the repression of Plexin-B1 protein expression. These agents, which can be identified and assessed according to the present invention, can be selected from the group formed by:
a) recombinant expression vectors expressing the Plexin-B1 protein. The vector can be introduced in vivo into the cells of the individual diagnosed with cancer; the expression vector can also be introduced into the cells ex vivo, yielding transformed cells which are subsequently introduced into the individual. Examples of recombinant expression vectors are chimeric viruses; examples of viral vectors useful for gene therapy include those vectors derived from adenovirus, herpes virus, vaccinia or any other virus whose genetic material is RNA, such as avian or murine retroviruses. In these vectors, the plexin-B1 gene can be bound to a specific gene expression promoter of the tissue affected by the cancer, for example, the kidney, such that the expression of the vector in other tissues where it is not necessary is prevented. Other examples of systems for introducing the plexin-B1 into the interior of tumor cells by means of a vehicle are colloidal dispersion systems; examples of colloidal dispersion systems include macromolecular complexes, nanocapsules, microspheres, beads and lipid systems such as oil-in-water emulsions, mycelia, mixed mycelia and liposomes.
b) cytotoxic agents such as toxins, molecules with radioactive atoms,. or chemotherapeutic agents, included among which are, with no limit, small organic and inorganic molecules, peptides, phosphopeptides, anti-sense molecules, ribozymes, triple helix molecules, etc., inhibiting the carcinogenic effects of Plexin-B1 protein expression and/or activity repression, and
c) Plexin-B1 protein agonist compounds which induce, mimic or replace one or more of the functions of the Plexin-B1 protein.

A further aspect the invention is constituted of the use of the agents characterized in that they induce Plexin-B1 protein expression and/or activity, as well as the use of the Plexin-B1 protein itself, in treating renal cancer. A preferred agent is the Plexin-B1 protein itself. These agents, in particular the Plexin-B1 protein, may be used in the form of pharmaceutically acceptable salts, derivatives or prodrugs. These terms refer to any pharmaceutically acceptable salt, ester, solvate, hydrate or any other compound which, upon administration to the recipient is capable of providing (directly or indirectly) a compound as describe herein. However, it will be appreciated that non-pharmaceutically acceptable salts also fall within the scope of the invention since those may be useful in the preparation of pharmaceutically acceptable salts. The preparation of salts, prodrugs and derivatives can be carried out by methods known in the art.

Also constituting an aspect of the present invention is a pharmaceutical composition comprising a therapeutically effective amount of one or several agents of those previously mentioned, or of the Plexin-B1 protein itself, along with one or more excipients and/transporting substances. Furthermore, said composition can contain any other active ingredient inducing, mimicking or replacing one or more of the functions of the Plexin-B1 protein. The excipients, transporting substances and auxiliary substances must be pharmaceutically and pharmacologically tolerable, so that they can be combined with other components of the formulation or preparation and do not cause adverse effects in the treated organism. The pharmaceutical compositions or formulations include those which are suitable for oral or parenteral (including subcutaneous, intradermal, intramuscular or intravenous) administration, although the best administration route depends on the condition of the patient. The formulations can be in the form of single doses. The formulations are prepared according to methods known in the pharmacology field. The active substance amounts to administer may vary according to the particularities of the therapy.

An additional aspect of this invention is the use of nucleotide or peptide sequences derived from the plexin-B1 gene as targets or tools for developing drugs for treating renal cancer in an individual.

A final aspect of the present invention consists in a kit for carrying out the present invention. Thus, an embodiment of the present invention provides a kit that comprises an antibody that specifically recognizes the Plexin-B1 protein and a carrier in suitable packing. In another embodiment the kit of the invention comprises a primer pair designed to specifically amplify a nucleic acid having a sequence that is specific to the plexin-B1 gene. The sequence of the primer pair can be determined from the sequence of the corresponding plexin-B1 gene by employing bioinformatic tools. The sequence of said primer pair is preferably selected from SEQ ID NO: 1 and SEQ ID NO: 2. These kits can be employed to detect the presence of renal cancer in an individual, to determine the stage or severity of this cancer in an individual or to monitor the effect of the therapy administered to the individual with this cancer.

The following examples illustrate the invention.

### Example 1.

### Differential analysis of plexin-B1 gene expression in kidney tissue samples using the Human Genome U95 DNA arrays microarrays.

### 1.1. Materials and Methods

Microarrays. The microarrays used were the GeneChip Test 3 microarray (Affymetrix, Santa Clara), which allow testing the quality of the RNA, prior to the expression analysis with the GeneChip Human Genome U95A array (Afymetrix, Santa Clara), representing 12,000 complete sequences of recorded genes; the plexin-B1 gene (plexin B1) is represented in the microarray by the 33783_at probe set of Affymetrix, which are sense oligonucleotides of 25 nucleotides in length, designed on the basis for the Hs.200480 sequence of Unigene, or AB007867 of GeneBank (Table 1).

TABLE 1
Description of the probes corresponding to the 33783_at probe set

| Consecutive | Zone of the | Sequence of | Probe position |
|---|---|---|---|
| order of the | interrogated | the probe | in mRNA |
| probes | reference sequence | (5′→3′) | sequence |
| 1 | 6720 | SEQ ID NO: 3 | 6508 |
| 2 | 6757 | SEQ ID NO: 4 | 6545 |
| 3 | 6775 | SEQ ID NO: 5 | 6563 |
| 4 | 6777 | SEQ ID NO: 6 | 6565 |
| 5 | 6863 | SEQ ID NO: 7 | 6651 |
| 6 | 6871 | SEQ ID NO: 8 | 6659 |
| 7 | 6882 | SEQ ID NO: 9 | 6670 |
| 8 | 6916 | SEQ ID NO: 10 | 6704 |
| 9 | 6918 | SEQ ID NO: 11 | 6706 |
| 10 | 7021 | SEQ ID NO: 12 | 6809 |
| 11 | 7024 | SEQ ID NO: 13 | 6812 |
| 12 | 7055 | SEQ ID NO: 14 | 6843 |
| 13 | 7057 | SEQ ID NO: 15 | 6845 |
| 14 | 7210 | SEQ ID NO: 16 | 6997 |
| 15 | 7222 | SEQ ID NO: 17 | 7009 |
| 16 | 7274 | SEQ ID NO: 18 | 7061 |

Samples. The kidney samples studied were from biopsies, obtained by surgical resection, of 7 individuals with renal oncocytoma (n=3), conventional renal carcinoma (n=3), chromophobe renal carcinoma (n=1). Neoplastic tissue and, as a negative control, non-neoplastic tissue were collected from each individual. All the samples were histologically classified in the Pathological Anatomy of the Hospital Universitario Marques de Valdecilla, the same hospital where the samples had been collected, following the precepts of the Declaration of Helsinki. The samples were frozen in liquid nitrogen immediately after extraction thereof and were kept at −80° C. until they were analyzed.

### GeneChip Gene Expression Analysis

The analysis was carried out with total RNA from) equimolar mixtures (pools) of total RNA from a set of 3 neoplastic kidney tissue samples from 3 individuals with renal oncocytoma (pool 1), equimolar mixtures of total RNA from a set of 3 neoplastic kidney tissue samples from 3 individuals with conventional renal carcinoma (Pool 2), total RNA from 1 neoplastic kidney tissue sample from 1 individual with chromophobe renal carcinoma (Sample #1), and, as a negative control, with equimolar mixtures of total RNA from a set (n=5) of non-neoplastic kidney tissue samples from individuates with renal oncocytoma, conventional renal carcinoma and chromophobe renal carcinoma (Pool 3) (Table 2).

TABLE 2
Description of the kidney tissue samples analyzed

| | Renal | Conventional renal | Chromophobe renal |
|---|---|---|---|
| | oncocytoma | carcinoma | Carcinoma |
| Neoplastic | Pool 1 | Pool 2 | #1 |
| tissue | (n = 3) | (n = 3) | (n = 1) |
| samples | | | |

| | |
|---|---|
| Non-neoplastic | Pool 3 |
| tissue | (n = 5) |
| samples | |

cRNA Synthesis

The total RNA of each one of the biopsies was obtained by homogenizing the tissue in TRIzol® Reagent (Life Technologies), following the supplier recommendations. The resulting total RNA was cleaned with the Rneasy (QIAGEN) kit (Chomczynski P. et al., Anal. Biochem., 1987, 162: 156; Chomczynski P., Biotechniques, 1993, 15: 532). 10 μg of every total RNA preparation were used as starting material for the synthesis of the first cDNA strand with the SuperScript™ II RNase reverse transcriptase enzyme (Life Technologics), using, an oligo-dT oligonucleotide containing the sequence of the promoter of the RNA polymerase of the T7 phage, as a primer. The second cDNA strand was synthesized using E. coli DNA polymerase I enzymes (Invitrogen Life Technologies), E. coli DNA ligase (Invitrogen Life Technologies), E. coli Rnase H (Invitrogen Life Technologies), and DNA polymerase of T4 phage (Invitrogen Life Technologies). The cRNA marked with biotin was synthesized using the ENZO BioArray™ HighYield™ Transcript Labeling Kit (Enzo Diagnostics Inc). After the in vitro transcription, the unincorporated nucleotides were removed by using the Rneasy columns (QIAGEN).

### Array Hybridization and Scanning

15 μg of each biotinylated cRNA were fragmented at 94° C. for 35 minutes in a buffer solution containing 40 mM Tris-Acetate (pH 8.1), 100 mM KOAc and 30 mM MgOAc. The fragmented cRNA was mixed with a hybridization buffer (100 mM MES, 1M NaCl, 20 mM EDTA, 0.01% Tween 20) and was heated at 99° for 5 minutes and then at 45° for 5 minutes, to then be loaded in the Affymetrix array. The first array in which hybridization was carried out was Affymetrix Test 3. This array allows testing the quality of the RNA prior to expression analysis in the Affymetrix® GeneChip® Human Genome 95 A (HG-U95A).

For hybridization, the arrays were incubated in a rotary oven at 45° for 16 hours and with a constant rotation of 60 rpm.

Washing and staining of each array were carried out in the Affymetrix® Fluid Station. A washing and staining program was used which included:
10×2 washing cycles with SSPE-T 6×(0.9 m NaCl, 60 mM NaH₂PO4, 6 mM EDTA, 0.01% Tween 20) at 25°,
4×15 cycles with 0.1 mM MES, 0.1M NaCl, 0.01% Tween 20 at 50°,
Biotinylated cRNA staining with a streptavidin-phycoerythrin conjugate (10 μg/ml Molecular Probes)
10×4 washing cycles with SSPE-T at 25°
Staining with an anti-streptavidin antibody for 10 minutes
Staining a streptavidin-phycoerythrin conjugate (1 mg/ml, Molecular Probes) for 10 minutes
15×4 washing cycles with SSPE-T at 30°

The arrays were scanned at 560 nm using a confocal microscope which uses laser emission (Agilent GeneArray Scanner). Intensity reading analysis was carried out with the Microarray Suite 5.0 software. To compare the arrays, they were scaled up to a total intensity of 100.

### 1.2. Results

Differential plexin-B1 gene expression analysis in the neoplastic tissues compared to the control, non-neoplastic tissues, was carried out starting from the array comparison data obtained using the Affymetrix software. The parameters considered (in the order in which they appear on the list) were: i) Detection. This indicates if the transcript is Present (P), Absent (A) or Marginal (M), ii) Change: This indicates if the expression of a certain transcript Increases (I), Decreases (D), experiences No Change (NC), Marginally Increases (MI), or Marginally Decreases (MD), iii) Signal Log Ratio (SLR): This indicates the expression change level between the baseline (control) and a test sample. This change is expressed as log₂ of the ratio (fold change or number of times the expression of the gene increases or is repressed in the tumor test sample compared to the health control sample). An SLR value of 1 (equivalent to a fold change of 2) is considered significant for transcripts having an expression that increases compared to the control, and an SLR value of −1 is considered significant for transcripts having an expression that decreases compared to the control.

Differential plexin-B1 gene expression analysis in the tumor stages with regard to the control showed that the plexin-B1 gene expression levels were repressed in the neoplastic kidney tissue with regard to the non-neoplastic tissue, and that the greater the malignity of the analyzed tumor, the greater this repression became: the repression level was greater than 2 fold (SLR <−1) in renal oncocytoma (benign) biopsies, greater than 4 fold (SLR<−2) in conventional renal carcinoma (malignant) biopsies and greater than 8 fold (SLR<−3) in chromophobe renal carcinoma (higher degree of malignity) biopsies (Table 3).

TABLE 3
Results obtained for Plexin B1. Acc. N. AB007867
Oncocytoma vs. Control

| | | Detection | SLR | CHANGE |
|---|---|---|---|---|
| Affy Seq | Detection Pool 3 | pool 1 | pool 1 vs. Pool 3 | pool 1 vs. Pool 3 |
| 33783_at | P | A | −1, 2 | D |

Conventional carcinoma vs. Control

| | | Detection | SLR | CHANGE |
|---|---|---|---|---|
| Affy Seq | Detection Pool 3 | Pool 2 | Pool 2 vs. Pool 3 | Pool 2 vs. Pool 3 |
| 33783_at | P | A | −2, 4 | D |

Chromophobe carcinoma vs. Control

| | | Detection | SLR | CHANGE |
|---|---|---|---|---|
| Affy Seq | Detection Pool 3 | Samples #1 | #1 vs. Pool 3 | #1 vs. Pool 3 |
| 33783_at | P | A | −3, 6 | D |

### Example 2

### Differential plexin-B1 gene expression analysis in kidney tissue samples using the quantitative real time RT-PCR technique.

### 2.1. Materials and Methods

To determine the plexin-B1 gene expression levels, quantitative real time RT-PCR was carried out in a 7000 Sequence Detection System using an SYBR green PCR master mix (Applied-Biosystems, Foster City, USA). The total RNA was extracted from individual biopsies using Trizol™ Reagent (Life Technologies, USA), following the instructions recommended by the manufacturer; this total RNA was then purified with the Qiagen Mini Kit spin columns (Qiagen, USA.). The integrity of the RNA was confirmed by electrophoresis in 1% agarose gel and the RNA was spectrophotometrically quantified. Then, 5 μg pf total RNA were digested with DNase I (1.2 uu/μg RNA) and the cDNA was synthesized following the following protocol: One μg of RNA treated with DNAse was mixed, in a total volume of 20 μl, with SuperScript™ Rnase H-Reverse Transcriptase (Invitrogen, USA.) (400 units/μg RNA) and with 100 pmols of oligo-dT. The synthesized cDNA was amplified using specific primers of the human plexin-B1 gene (SEQ ID NO: 1, and SEQ ID NO: 2), and specific primers of the human glyceraldehyde-3-phosphate-dehydrogenase (gapdh) gene. Then, the ratio between the abundance of transcribed plexin-B1 mRNAs and the abundance of the gapdh transcripts was calculated, as the relative measurement of gene expression: 2ⁿ, where n is the value Cₜ (threshold cycle) of gapdh minus the plexin-B1 Cₜ value, and the data of the ratio was normalized based on the value of the sample with the lowest plexin-B1 expression level. The specificity of the PCR products was determined by denaturation curve analysis. A standard curve was constructed for each gene sequence, carried out with serial dilutions of cDNA; the mold cDNA concentrations were given the arbitrary values 10, 5, 2.5, 1.25, 0.625, 0.3125 and 0.15625 for the reactions on the standard curve. The real time PCR reactions were prepared using the LightCycler-FastStart DNA master SYBR Green I kit (Roche, USA.), following the instructions of the manufacturer. The amplification program consisted of 1 cycle of 95° C. for 1 min (hot start) followed by 45 cycles of 95° C. (denaturation) for 10 seconds, 60° C. (annealing) for 5 seconds, 72° C. (amplification and acquisition) for 10 seconds. The denaturation curve analysis program consisted of one cycle of a 95° C., 65° C. pulse for 15 seconds, and a 95° C. pulse during the amplification and acquisition step.

Amplification efficiency was calculated for each PCR reaction based on the standard curve data, using the equation:

E=10^{-1/slope} [1]

where E is the amplification efficiency. The ratio of the gene expression values were determined by the equation:

$$Ratio = \frac{{E}_{target}^{- ( Cp target control - Cp target sample )}}{{E}_{reference}^{- ( Cp reference control - Cp reference sample )}} \qquad [ 2 ]$$

where E is the amplification efficacy, Cp is the cross point, target is plexin-B1, reference is GAPDH, control is the healthy sample and sample is the tumor sample.

### 2.2. Results

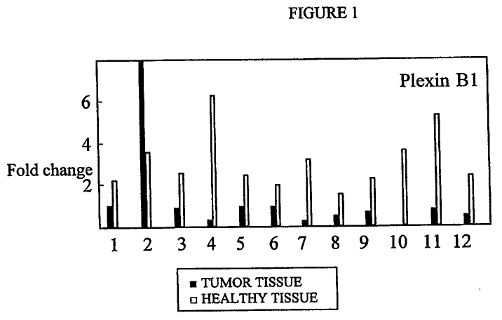

Amplification and denaturation curves of the target gene (plexin-B1) and of the reference gene (gadph) were constructed; on the denaturation curve, a single defined peak was observed at the temperature corresponding to the denaturation temperature of each product, which indicated that the reactions were specific (data not shown). Each samplewas analyzed in triplicate to calculate the relative change in the gene expression levels using equations [1] and [2] described in the previous section. The changes in the plexin-B1 gene expression levels are shown in FIG. 1. Only one out of the neoplastic samples analyzed showed gene expression levels exceeding the levels in the non-neoplastic levels; in the other cases, which included conventional, papillary and chromophobe renal carcinoma (table 4), expression levels were clearly repressed with regard to the samples from non-neoplastic tissue (FIG. 1).

TABLE 4
Clinicopathological data of the renal neoplasms analyzed

| Case | Age | Sex | Histological Diagnosis | Stage | Evolution |
|---|---|---|---|---|---|
| 1 | 58 | Male | Conventional RC | II | A&H (28 months) |
| 2 | 80 | Male | Conventional RC | II | A&H (16 months) |
| 3 | 78 | Male | Conventional RC | II | A&H (20 months) |
| 4 | 38 | Male | Conventional RC | II | A&H (24 months) |
| 5 | 68 | Male | Conventional RC | II | A&H (24 months) |
| 6 | 76 | Female | Conventional RC | II | A&H (22 months) |
| 7 | 51 | Male | Conventional RC | III | DDD (15 months) |
| 8 | 45 | Male | Conventional RC | III | DDD (11 months) |
| 9 | 57 | Male | Chromophobe RC | II | A&H (25 months) |
| 10 | 55 | Male | Papillary RC | II | A&H (24 months) |
| | | | (low grade) | | |
| 11 | 69 | Male | Papillary RC | III | A&H (18 months) |
| | | | (high grade) | | |
| 12 | 79 | Male | Renal oncocytoma | II | A&H (26 months) |

RC: Renal carcinoma
A&H (n months): Alive and Healthy, n months after diagnosis.
DDD (n months): Death due to disease, n months after diagnosis.

### Example 3

### Proliferation assays in parental and transfected ACHN human renal adenocarcinoma cell line.

### 3.1. Materials and Methods

Transfection and Cell Cloning

ACHN human renal adenocarcinoma cells were obtained from the European Collection of Cell Cultures (ECACC No. 88100508), and cultured in DMEM medium supplemented with 10% FCS, 2 mM glutamine and antibiotics (all from Gibco/BRL, Paisley, UK). For transfection, 10⁶ cells were plated in 10-cm tissue culture dishes (Corning, Corning, USA) and transfected 24 h later with 10 μg of plasmid DNA by the calcium phosphate method. The eukaryotic expression vector pCEFL/plexinB1 which contains a neomycin resistance expression cassette, was used to drive full-length plexinB1 expression from the EF1 promoter. As a control, vector pMEX was used (Katzav S, Martin-Zanca D, Barbacid M, 1989 EMBO J, vol 8, pp. 2283-2290), which also contains the neomycin resistance cassette. 48 h after transfection, cells were transferred to 96-well plates (Corning) and 1 mg/ml Geneticin (Gibco/BRL) was added to the culture medium to select for stably transfected clones. After expansion in 75-cm² flasks (Corning), plexinB1 expression in Geneticin-resistant clones was verified by standard RT-PCR methods with primers located at the 3′ end of the coding sequence (SEQ ID NO: 19 [forward] and SEQ ID NO: 20 [reverse]). As a control, RT-PCR was performed on the housekeeping gene rib 110 (SEQ ID NO: 21 [forward] and SEQ ID NO: 22 [reverse]).

MTT (cell proliferation) Assay

500 parental ACHN, ACHN/pMEX (AM) or ACHN/plexinB1 (AP) cells were seeded per well in 200 μl of culture medium in 96-well plates (Corning) and in replicates of 8. After incubation for 24, 48, 72 or 96 h, 20 μl of a stock solution of MTT (Sigma, Steinheim, Germany) was added to the cultures for a final concentration of 50 μg/ml. Upon incubation for 2 h at 37C, the medium was decanted and 200 μl DMSO were added to each well. Plates were incubated for 30 min at room temperature to allow dissolution of MTU crystals. Optical density (OD) readings were taken on a Labsystems (Ashford, UK) Multiscan RC plate reader fitted with 570 nm and 650 nm filters. Absorbance was determined by averaging the difference between OD₅₇₀ and OD₆₅₀ readings. Regression curves for cell proliferation data were calculated and plotted with Microsoft Excel 2000 software.

### 3.2 Results

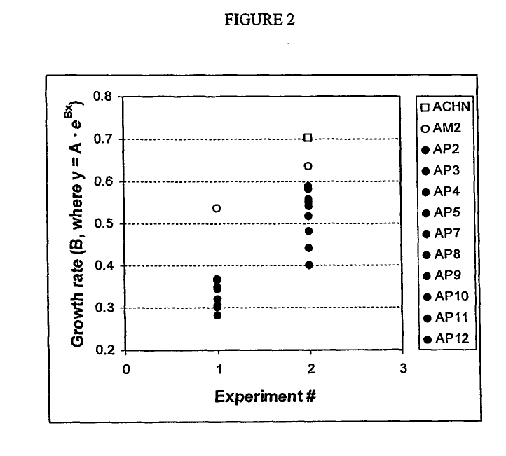

All plexinB1-expressing clones (AP2-AP12) proliferate more slowly than the control clone (AM2) or parental ACHN cells (Table 5 and FIG. 2) in both experiments.

TABLE 5
Values for the growth rate in 2 independent determinations

| | Exp. #1 | Exp. #2 |
|---|---|---|
| ACHN | — | 0.7019 |
| AM2 | 0.5346 | 0.6366 |
| AP2 | 0.3632 | 0.5581 |
| AP3 | 0.3664 | 0.5876 |
| AP4 | 0.3678 | 0.5407 |
| AP5 | 0.3179 | 0.4007 |
| AP7 | 0.3430 | 0.5509 |
| AP8 | 0.3484 | 0.4802 |
| AP9 | 0.2817 | 0.4400 |
| AP10 | 0.3475 | 0.5169 |
| AP11 | 0.2998 | 0.5821 |
| AP12 | 0.3047 | — |

### 3.3 Discussion

The MTT (cell proliferation) assays have shown that the proliferation rate of ACHN human renal adenocarcinoma cells is significantly lower when those cells are expressing Plexin B1. These results suggest that the expression of Plexin B1 causes a decrease in cell proliferation. These functional data, altogether with data at the molecular expression level, from DNA microarrays, Q-PCR and Inmunohistochemistry, suggest an antiproliferative role for Plexin-B1 in renal carcinomas.

### Example 4

### Analysis of protein expression in tissue samples using tissue arrays

### 4.1. Materials and Methods

Fixed paraffin-embedded tumour samples from the pathology archives of the Hospital Universitario Marqués de Valdecilla were sectioned and arrayed on glass slides. In total 51 cases of adult renal cell carcinoma [26 cases of clear cell (conventional) renal cell carcinoma and 25 cases of papillary renal cell carcinoma] from nephrectomy specimens and 6 healthy kidney samples were examined by immuno-histochemical staining. All paraffin-embedded donor tissue blocks were sampled with 1.00-mm punchers using a Beecher tissue microarray instrument (Beecher Instruments Inc. Sun Prairie, Wis., USA). Paraffin tissue array blocks containing arrayed core cylinders were subjected to routine staining with hematoxylin and eosin followed by immunohistochemical staining for the Plexin B1. Briefly, antigen retrieval was performed by boiling sections in citric acid buffer in a pressure cooker for 180 sec. Next, a 1:100 dilution of a rabbit polyclonal serum raised against a KLH-plexinB1 peptide conjugate (SEQ ID NO. 23, residues 1113-1127) was incubated with the specimen. This was followed by visualization with the Dako EnVision™+ system (Dako, Glostrup, Denmark), which is based on the use of a secondary goat-anti-rabbit antibody conjugated to a peroxidase-linked polymer followed by a chromogen substrate (diaminobenzidine). The immunostaining process was performed on a Techmate 500-220 automated immunostainer (Biotek, Santa Barbara, Calif., USA).

To reduce interobserver variability in the histopathological evaluation of the antibody-stained specimens three independent pathologists from the Pathological- Anatomy Department of the University Hospital Marques de Valdecilla evaluated staining patterns and scoring criteria were agreed. Positive staining of Plexin B1 was defined as a coarse cytoplasmic membrane reactivity. Immunohistochemistry was considered negative in cases where staining was absent or which showed weak staining (<5% of cells in a given section).

### 4.2. Results

The specificity of the immunohistochemical staining is supported by the following findings: (1) Staining is tissue specific, i.e. present in healthy tissue but absent in a significant part of tumor tissues, in agreement with microarray and Q-PCR data. (2) Staining is positive for renal tubule cells, cells in which the tumors under study originate, but not for other cell types such as vascular cells, mesenchymal cells or the inflammatory infiltrate. (3) Staining is present on the cell membrane and the cytoplasm, without any precipitates, but is absent from the nucleus. (4) Staining is blocked by addition of the immunizing peptide.

Of the adult renal cell carcinoma sections that were analysed immunohistochemically a positive reaction with the antibody specific for Plexin B1 was positive in 19% of clear cell (conventional) renal cell carcinoma sections, 40% of papillary renal cell carcinoma compared to the 83% of healthy positive sample (table 6).

TABLE 6
Results of Immunohistochemical Staining

| | Total | | | % of |
|---|---|---|---|---|
| | n° of | Positive | Negative | positive |
| | samples | cases | cases | cases* |
| clear cell (conventional) | 26 | 5 | 21 | 19% |
| renal cell carcinoma | | | | |
| papillary renal cell carcinoma | 25 | 10 | 15 | 40% |
| healthy kidney samples | 6 | 5 | 1 | 83% |

*Positive/(Positive + Negative) × 100

### 4.3. Discussion

Consistent with the Plexin B1 gene expression data in renal cell carcinomas versus healthy kidney, sections classified as carcinomas showed a lower percentage of positive staining with labelled Plexin B1 antibody (19% and 40%) than sections corresponding to healthy kidney (83%). These results show that Plexin B1 protein is not expressed in a high percentage of renal cell carcinomas.

1. An in vitro method for detecting the presence of renal cancer in an individual, to determine a stage or severity of said renal cancer in an individual or to monitor the effect of a therapy administered to said individual with said renal cancer, said method comprising:
a) detection and/or quantification of Plexin-B1 protein, of mRNA of a plexin-B1 gene, or of corresponding cDNA in a sample of said individual, and
b) comparison of an amount of Plexin-B1 protein, of an amount of plexin-B1 gene mRNA or of an amount of the corresponding cDNA detected in a sample from said individual, with their normal reference values.

2. The in vitro method according to claim 1, wherein said sample comprises a kidney tissue sample.

3. The in vitro method according to claim 1, as employed to detect the presence of renal cancer in said individual.

4. The in vitro method according to claim 2, further comprising analyzing said kidney tissue sample by a method, comprising nephrectomy.

5. The in vitro method according to claim 1, wherein said sample is a urine, blood, plasma, serum, pleural fluid, ascitic fluid, synovial fluid, bile, semen, gastric juice or cerebrospinal fluid sample.

6. The in vitro method according to claim 1, wherein said sample has been obtained from an individual who has not previously been diagnosed with renal cancer.

7. The in vitro method according to claim 1, wherein said sample has been obtained from an individual who has previously been diagnosed with renal cancer.

8. The in vitro method according to claim 1, wherein said sample has been obtained from an individual undergoing treatment, or who has been treated previously, for renal cancer.

9. The in vitro method according to claim 1, comprising extracting the sample, either for obtaining a protein extract or for obtaining an extract of total RNA.

10. The in vitro method according to claim 1, characterized in that the detection and/or quantification of the Plexin-B1 protein comprises a first step, in which the protein extract of the sample is contacted with a composition of one or more specific antibodies against one or more epitopes of the Plexin-B1 protein, and a second step, in which complexes formed by the antibodies and the Plexin-B1 protein are quantified.

11. The in vitro method according to claim 10, characterized in that said antibodies comprise antibodies selected from among monoclonal antibodies, polyclonal antibodies, either intact or recombinant fragments thereof, combined antibodies and Fab or scFv antibody fragments, specific against the Plexin-B1 protein; he wherein said antibodies are human, humanized or of a non-human origin.

12. The in vitro method according to claim 10, characterized in that in the detection and/or quantification of the complexes formed by the antibodies and the Plexin-B1 protein, comprises use of a technique selected from the group consisting of: Western-blot, ELISA (Enzyne-Linked Immunosorbent Assay), RIA (Radioimmunoassay), Competitive EIA (Competitive Enzyme Immunoassay), DAS-ELISA (Double antibody Sandwich-ELISA), immunocytochemical and immunohistochemical techniques, techniques based on the use of biochips or protein microarrays that include specific antibodies, assays based on precipitation with colloidal gold, affinity chromatography techniques, ligand binding assays and lectin binding assays.

13. The in vitro method according to claim 1, characterized in that the detection and/or quantification either of the mRNA or of the corresponding cDNA of the plexin-B1 gene comprises a first step of amplification of the mRNA that is present in the an extract of total RNA from said sample, or of the corresponding cDNA synthesized by reverse transcription of the mRNA, to yield an amplification product; and a second step of quantification of the amplification product from either the mRNA or the cDNA of the plexin-B1 gene.

14. The in vitro method according to claim 13, characterized in that the amplification is performed qualitatively or quantitatively by means of RT-PCR using primer oligonucleotides, where the sequences of the primer oligonucleotides used to amplify the sequence of the plexin-B1 gene are SEQ ID NO. 1 and SEQ ID NO. 2.

15. The in vitro method according to claim 1, characterized in that the detection and/or quantification comprises use of specific probes of the mRNA or of the corresponding cDNA of the plexin-B1 gene.

16. The in vitro method according to claim 1, comprising detection of the mRNA by real time quantitative RT-PCR (Q-PCR).

17. A method of diagnosis and/or monitoring of an individual actually or potentially having renal cancer, said method comprising use of a nucleotide or peptide derivative of the plexin-B1 gene to detect in vitro the presence of renal cancer in an individual, to determine in vitro the stage or severity of said cancer in the individual or to monitor in vitro the effect of the therapy administered to an individual having said cancer.

18. An in vitro method for identifying and assessing the efficacy of compounds for renal cancer therapy, comprising:
a) contacting an immortalized kidney cell culture with a candidate compound under conditions and for a time enabling them to interact,
b) detecting and quantifying plexin-B1 gene or Plexin-B1 protein expression levels in said immortalized kidney cell culture, and
c) comparing said expression levels with those of immortalized kidney cell control cultures not treated with the candidate compound.

19. A method of at least one of search, identification, development and assessment of the efficacy of compounds for renal cancer therapy, said method comprising use of nucleotide or peptide sequences derived from the plexin-B1 gene.

20. A recombinant expression vector adapted for expression of the Plexin-B1 protein.

21. A method for treating renal cancer, comprising administration of an effective amount of a recombinant expression vector according to claim 19.

22. A method for treating renal cancer, comprising administration of an effective amount of Plexin B1 protein.

23. A method for treating renal cancer, comprising administration of an effective amount of an agent that induces Plexin-B1 protein expression and/or activity, or that inhibits the carcinogenic effects of the repression of Plexin-B1 protein expression.

24. The method for treating renal cancer according to claim 23, wherein said agent is selected from the group consisting of:
a) agents that inhibit carcinogenic effects of repression of Plexin-B1 protein expression and/or activity and
b) Plexin-B1 protein agonist compounds that induce, mimic or replace one or more of the functions of the Plexin-B1 protein.

25. A pharmaceutical composition comprising a therapeutically effective amount of an agent that inhibits carcinogenic effect of the repression of Plexin-B1 protein, and a pharmaceutically acceptable excipient.

26. The pharmaceutical composition according to claim 25, wherein the agent is selected from the group consisting of:
(a) recombinant vectors expressing the Plexin-B1 protein;
(b) cytotoxic agents; and
(c) Plexin-B1 protein agonist compounds that induce, mimic or replace one or more of the functions of the Plexin-B1 protein.

27. The pharmaceutical composition according to claim 26, wherein the agent comprises a Plexin-B1 protein.

28. The pharmaceutical composition according to claim 26 wherein the agent comprises a recombinant expression vector adapted to express the Plexin-B1 protein.

29. A kit to detect presence of renal cancer in an individual, to determine a stage of the severity of said cancer in an individual, or to monitor effect of a therapy administered to an individual with said cancer, said kit comprising an antibody that specifically recognizes the Plexin-B1 protein, and a carrier, in packaged form.

30. A kit to detect the presence of renal cancer in an individual, to determine a stage of severity of said cancer in an individual, or to monitor effect of a therapy administered to an individual with said cancer, said kit comprising a primer pair adapted to specifically amplify a nucleic acid having a sequence that is specific to the plexin-B1 gene.

31. A kit according to claim 30, wherein the sequence of the primer pair is selected from SEQ ID NO. 1 and SEQ ID NO. 2.

32. A method of diagnosis and/or monitoring of an individual actually or potentially having renal cancer, said method comprising use of a kit selected from the group consisting of:
kits comprising an antibody that specifically recognizes the Plexin-B1 protein and a carrier; and
kits comprising a primer pair adapted to specifically amplify a nucleic acid having a sequence that is specific to the plexin-B1 gene.

34. The method according to claim 32, wherein the kit comprises a primer pair adapted to to specifically amplify a nucleic acid having a sequence that is specific to the plexin-B1 gene, wherein the sequence of the primer pair is selected from SEQ ID NO 1 and SEQ ID NO 2.

35. The pharmaceutical composition of claim 25, further comprising a substance inducing Plexin-B1 protein function.
